(19) United States
(12) Patent Application Publication
(10) Pub. No.: US 2002/0099428 A1
(43) Pub. Date: Jul. 25, 2002

(54) Position-controlled heat delivery catheter

(76) Inventor: Leon Kaufman, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/771,024

(22) Filed: Jan. 25, 2001

(51) Int. Cl. A61B 18/18

(52) U.S. Cl. 607/101; 606/41; 606/27

(57) ABSTRACT

Methods and devices for delivering heat to a target region within a body lumen. In one aspect, the present invention provides a method of delivering heat to a body lumen. The method comprises positioning a catheter comprising an electrode array in the body lumen and selectively delivering a current to fewer than all of the electrodes in the electrode array to heat a target region of the body lumen.

### BACKGROUND OF THE INVENTION

The present invention relates generally to medical devices. More particularly, the present invention relates to the treatment of vulnerable plaque within a body lumen.

Coronary artery disease resulting from the build-up of atherosclerotic plaque and its subsequent rupture to form blood-flow blocking clots in the coronary arteries is the leading cause of death in the United States. The plaque build-up leads to a narrowing of the artery and as a consequence, reduces the blood flow to the myocardium (i.e., heart muscle tissue). Myocardial infarction, better known as a heart attack, can occur when the arterial plaque abruptly closes a vessel, causing complete cessation of blood flow to portions of the myocardium, or more likely the plaque ruptures, fissures, or erodes and produces a clot that blocks the vessel at that point or distally (downstream). Even if abrupt closure does not occur, blood flow may decrease resulting in chronically insufficient blood flow which can cause significant tissue damage over time.

Depending on the type of plaque present in the coronary arteries and other vessels, it may contain, among other components: inflammatory cells, smooth muscle cells, cholesterol, and/or fatty substances. These materials are usually trapped between the endothelium of the blood vessel and the underlying smooth muscle cells. Depending on various factors, including thickness, composition, and size of the deposited materials, the plaques can be characterized as stable or vulnerable. The plaque is normally covered by an end cap. However, under certain adverse conditions the cap can be disrupted, leading to the release of thrombogenic material which is capable of activating the clotting cascade and inducing coronary thrombosis. Such plaque is referred to as vulnerable plaque. The resulting thrombus caused by the vulnerable plaque can cause angina chest pain, acute myocardial infarction, stroke, or sudden coronary death. It has recently been proposed that plaque instability, rather than the degree of plaque build-up, should be the primary determining factor for treatment selection.

A variety of interventions have been proposed to treat coronary artery disease. For disseminated disease, the most effective treatment is usually coronary artery bypass grafting (CABG) where problematic lesions in the coronary arteries are bypassed using external grafts. In cases of less severe disease, pharmaceutical treatment is often sufficient. Finally, focused disease can often be treated intravascularly using a variety of catheter-based approaches, such as balloon angioplasty, atherectomy, radiation treatment, heat treatment, stenting, and sometimes combinations of these approaches. While each of the conventional treatments have provided some success in treating plaque in the body lumen, each of the conventional treatments has significant drawbacks. For example, U.S. Pat. No. 5,057,105 describes a hot tip catheter assembly comprising a heating tip for removing arterial plaque. Such a device can deliver heat to only one portion of the body lumen. If it is determined that the heating tip is not positioned adjacent the target tissue, the entire catheter must be moved, which increases the length of the procedure and consequently the danger to the patient. Furthermore, moving the tip to different locations risks the loss of registration with the detection device used to identify the areas in need of treatment.

For the above reasons, what is needed are devices and methods which can provide spatially controlled heat to the vulnerable plaque or other lesions while still being able to access the small and tortuous regions of the body lumen.

### DESCRIPTION OF BACKGROUND ART

U.S. patent application Ser. No. 09/754,103 filed Jan. 3, 2001, entitled “Intravascular Imaging Catheter,” U.S. patent application Ser. No. 09/754,074, filed Jan. 3, 2001, entitled “Position Sensitive Catheter,” and U.S. Pat. No. 09/754,822, filed Jan. 3, 2001, entitled “Position Sensitive Catheter having Scintillation Detectors” describe imaging catheters; and U.S. patent application Ser. No. 09/670,412, filed Sep. 26, 2000 describes methods and apparatus for Characterizing Lesions in Blood Vessels and Other Body Lumens, the complete disclosures of which are incorporated herein by reference.

Intravascular heat treatment of tissue is described in a number of patents including U.S. Pat. Nos. 6,113,593, 5,935,075, 5,906,636, 5,871,449, 5,057,105, and 5,009,655.

### SUMMARY OF THE INVENTION

The present invention provides improved intravascular heat treatment catheters and methods for their use. In particular, the catheters of the present invention are suitable for the delivery of a therapeutic heat treatment to early-stage, vulnerable coronary artery plaque disposed within the arteries and other blood vessels.

In one aspect, the present invention provides catheters which are adapted to deliver a current to fewer than all of the electrodes on the catheter.

In another aspect, the present invention provides heat treatment catheters comprising an electrode array that is attachable to a power source, such as an AC power source, a DC power source, an RF power source, or the like. The electrode array includes a plurality of position sensitive heat delivery electrodes that are configured to selectively deliver a therapeutic heat treatment to target portions of a body lumen, such as vulnerable plaque disposed within the body lumen.

In exemplary embodiments, the electrode array includes a plurality of cylindrical electrodes positioned on a distal portion of the catheter. In such embodiments, the electrodes may be disposed on an inner or outer sleeve positioned at the distal end of the catheter body or integrated directly on the interior or exterior surface of the catheter body. While it is possible to couple each of the electrodes with an individual energy conduit or conductor, the size and flexibility of the catheter body would be limited due to the large number of transmission lines extending through the catheter. Accordingly, in preferred embodiments, the electrode array can be coupled to a power supply through the single movable conductor. The movable conductor can be moved through the catheter body to a selectively communicate or contact individual electrodes of the electrode array so as to selectively deliver a current (or energy) from a power supply to at least one selected electrode contacted by the conductor and to deliver a heat treatment to a portion of the body lumen.

As used herein, “conductor” and “energy conduit” will generally be used to mean an element that is capable of delivering a current or an energy such as AC current, DC current, RF current, ultrasonic energy, optical light, or the like.

Optionally, the catheters may include a position sensitive detector assembly that can detect and characterize the vulnerable coronary artery plaque. Typically, the detectors will be able to identify and localize plaque-binding beta-emitting radiopharmaceuticals such that a heat treatment electrode or applicator can deliver the heat treatment to the targeted vulnerable plaque. The radiation detectors are typically integrated into an intravascular catheter with the heat treatment electrodes so that both the detectors and electrodes can be manipulated through the body lumen simultaneously. Consequently, only a limited amount of movement of the catheter, if any, is needed to position the array of electrodes adjacent the plaque.

In another aspect, the present invention provides methods of delivering a heat treatment to a portion of a body lumen. In particular methods, the present invention provides marking the vulnerable plaque with a radiopharmaceutical, imaging the marked vulnerable plaque, and delivering a therapeutic heat treatment to the localized vulnerable plaque.

In one exemplary method, a catheter comprising an electrode array is positioned in the body lumen. A current or energy is selectively delivered to one of the electrodes in the electrode array to heat a target region of the body lumen.

Optionally, a radiopharmaceutical or other marking agent can be delivered into the body lumen to mark the lesion in the body lumen. Thereafter, a detector can be positioned in the body lumen to localize the marked lesion. In most embodiments it is desirable to have a heat delivery assembly (e.g. electrode array) that is simultaneously delivered through the body lumen with a position-sensitive detector assembly. Consequently, after the detector(s) have localized the position of the marked lesion, the electrodes can be selectively activated without moving (or minimally moving) the catheter.

In yet another aspect, the present invention provides kits including any of the catheters, instructions for use, and a package. The catheters will generally be those as described herein and the instruction for use (IFU) will set forth any of the methods described herein. The package may be any conventional medical device packaging, including pouches, trays, boxes, tubes, or the like. The instructions for use will usually be printed on a separate piece of paper, but may also be printed in whole or in part on a portion of the packaging.

As will be appreciated by those versed in the art, while the present invention will find particular use in the diagnosis and treatment of lesions within blood vessels, the present invention will also be useful in a wide variety of diagnostic and therapeutic procedures. The methodology of plaque detection and treatment can be extended to the detection and treatment of malignancies following the administration of a metabolic or specific radiolabeled agents (e.g., labeled amino acids, labeled glucose, labeled nucleotides and nucleosides, or the like).

The above aspects and other features of the present invention may be more fully understood from the following detailed description, taken together with the accompanying drawings, and claims wherein similar reference characters refer to similar elements throughout the specification.

### BRIEF DESCRIPTION OF THE DRAWINGS

FIG. 1 illustrates a catheter comprising an array of heat delivery electrodes;

FIG. 2 is a cross sectional view of a detector array disposed within cylindrical electrodes;

FIG. 3 shows a detector array interspersed amongst an electrode array;

FIG. 4 illustrates cylindrical electrodes and a movable conductor of the present invention;

FIG. 5 illustrates broken cylindrical electrodes;

FIG. 6 is an end view of an RF antenna;

FIG. 7A illustrates a coaxial movable conductor of the present invention;

FIG. 7B illustrates a two strand movable conductor of the present invention;

FIG. 8A is a cross sectional view of a solid cylindrical electrode and a conductor with sliding contacts disposed approximately 180° apart from each other;

FIG. 8B is a cross sectional view of a broken cylindrical electrode and a conductor with sliding contacts disposed approximately 90° apart from each other;

FIG. 8C is a cross sectional view of a broken cylindrical electrode and a conductor with sliding contacts disposed approximately 45° apart from each other;

FIG. 8D illustrates an RF antenna and a conductor with sliding contacts disposed approximately 180° apart from each other;

FIG. 8E illustrates a cylinder having two longitudinal breaks and a conductor having sliding contacts disposed less than 180° apart from each other;

FIG. 8F illustrates a cylinder having more than two longitudinal breaks and a conductor having sliding contacts disposed less than 180° apart from each other for providing azimuthal control of heat delivery;

FIG. 8G illustrates a fixed conductor and a single sliding contact;

FIG. 8H illustrates a fixed conductor coupled to a cylinder having a longitudinal break;

FIG. 9A is a cross-sectional view of the electrode array having a fixed conductor and a sliding contact conductor;

FIG. 9B is a cross sectional end view of an electrode having a plurality of fixed conductors and a sliding conductor;

FIG. 10 illustrates the catheter positioned in a body lumen having a marked lesion;

FIG. 11 illustrates localizing the marked lesion;

FIG. 12 illustrates moving the conductor to a selected electrode adjacent the marked lesion; and

FIG. 13 illustrates a kit of the present invention.

### DESCRIPTION OF THE SPECIFIC EMBODIMENTS

The present invention provides improved methods and apparatus for treating body lumens, particularly for treating vulnerable plaque in blood vessels through the selective delivery of heat to a target region on a lumenal wall. In particular, the apparatus comprise energy effector arrays and positionable energy conductors for selectively energizing selected regions within the array. While the exemplary energy effector arrays will be an electrode array and the conductor will be an electrical conductor, it will be appreciated that alternative systems employing optical, ultrasonic, and other energy transmission and distribution means could be employed.

The methods of the present invention rely on treating target lumenal regions, particularly regions identified as vulnerable plaque within blood vessels, by selectively heating the targeted regions. In a preferred aspect of the present invention, the treating apparatus and methods will be combined with diagnostic apparatuses and methods that allow for identification and localization of the target regions. For example, the apparatus and methods for identifying and localizing vulnerable plaque as described in co-pending Patent Application Ser. Nos. 09/754,822, filed Jan. 3, 2001, 09/754,074, filed Jan. 3, 2001, and 09/754,103, filed Jan. 3, 2001, the full disclosures of which were previously incorporated herein by reference, may be incorporated into the treatment apparatus and methods of the present invention.

For example, vulnerable plaque may be localized by introducing a labeled marker, typically a radiopharmaceutical or radiolabeled marker with a binding agent, into the patient's blood vessel in such a way that the marker localizes within a lesion or target site which enables assessment of the type of plaque within the blood vessel. Introduction of the labeled marker can be systemic (e.g., oral ingestion, injection or infusion to the patient's blood circulation, and the like), through local delivery (e.g. by catheter delivery directly to a target site within the blood vessel), or a combination of systemic and local delivery.

After introduction of the marker to the patient, the marker will be taken up by the lesion (e.g., vulnerable plaque) at the target site and the amount of the marker, rate of uptake, distribution of the marker, or other marker characteristics can be analyzed to evaluate the severity of the lesion. The types of radio tracers and radio labels are more fully described in co-pending U.S. patent application Ser. No. 09/670,412, filed Sep. 26, 2000, and titled “Methods and Apparatus for Characterizing Lesions in Blood Vessels and Other Body Lumens,” licensed to the assignee of the present application, the complete disclosure of which was previously incorporated herein by reference.

Treatment, and optionally detection of vulnerable plaque or other lesions, within the body lumen can be performed in vivo using intraluminal catheters of the present invention. Radiation detection can thus be performed with a detector (or detector array) in close proximity to radiation localized within the lumen, and the measurement of radiation intensity can be very accurate and the process can remain robust. The lesions can then be heated by heating electrodes disposed on the same catheter. Preferably, the heating electrodes are disposed adjacent the detector array such that after localization of the marked lesion, little or no movement is needed to position the heating electrodes adjacent the marked lesion. In particular embodiments, the electrodes and detectors can be at least partially overlaid, interspersed amongst each other, positioned within each other, or the like.

The catheters according to the present invention will comprise catheter bodies adapted for intraluminal introduction through the body lumen to the target site. The dimensions and other physical characteristics of the catheter bodies will vary significantly depending on the body lumen which is to be accessed. In the exemplary case, the catheter bodies will typically be very flexible and suitable for introduction over a guidewire to a target site within the vasculature. In particular, catheters can be intended for “over-the-wire” introduction when a guidewire lumen extends fully through the catheter body (or separate guidewire lumen) or for “rapid exchange” introduction where the guidewire lumen extends only through a distal portion of the catheter body or the distal tip. In other cases, it may be possible to provide a fixed guidewire at the distal tip of the catheter or even dispense with the guidewire entirely. For convenience of illustration, guidewires will not be shown in all embodiments, but it should be appreciated that they can be incorporated into these embodiments.

Exemplary catheter bodies intended for intravascular introduction will typically have a length in the range from 10 cm to 200 cm and an outer diameter in the range from 1 French (0.33 mm: Fr.) to 24 Fr., usually from 1 Fr. to 20 Fr. In the case of coronary catheters, the length is typically in the range from 80 cm to 150 cm, the diameter is preferably below 8 Fr., more preferably below 6 Fr., and most preferably in the range from 2 Fr. to 4 Fr.

Catheter bodies will typically be composed of a polymer which is fabricated by conventional extrusion techniques. Suitable polymers include polyvinylchloride, polyurethanes, polyesters, polyolefins, polytetrafluoroethylenes (PTFE), silicone rubbers, polyamides, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more lumens being provided when desired. The catheter diameter can be modified by heat expansion and shrinkage using conventional techniques. The resulting catheters will thus be suitable for introduction to the vascular system, often the coronary arteries, by conventional techniques.

The catheters of the present invention can include an electrode array comprising a plurality of heating electrodes that are positioned to heat a portion of the body lumen, and more specifically to the vulnerable plaque. In exemplary embodiments, the heating electrodes will be cylindrical so as to provide a therapeutic heat treatment to the inner circumference of the body lumen. The cylindrical electrodes will typically be disposed along a common axis and each electrode will typically have a length between approximately 1 mm and 1 cm. In most embodiments, the entire array of electrodes will include between approximately 5 and 60 electrodes and will cover an overall length between approximately 1 cm and 5 cm.

In other embodiments, the heating electrodes can be a broken cylinder, pads, or the like. The heating electrodes are composed of an electrically and thermally conductive material that can deliver the heat treatment to the vulnerable plaque. By altering the electrode configuration, the electrode array can be adapted to control the axial and/or azimuthal delivery of the heating.

Suitable materials for the electrodes include aluminum, copper, platinum, tungsten, silver, gold, tantalum, their alloys, stainless steel and the like.

Optionally, the catheters of the present invention will have an array of position-sensitive radiation detectors that are capable of detecting ionizing radiation from a radio-isotopic label within a distance between approximately 0.1 cm and 1 cm from the radio label. The radiation detectors can be sized and configured to be able to image a length of the body lumen between approximately 1 cm and 5 cm. The radiation detectors can be coupled to a delay line to reduce the number of transmission lines running through the catheter body. It will be appreciated by those of ordinary skill in the art that, while radiation detectors are preferred, other types of imaging detectors can be incorporated into the catheters of the present invention.

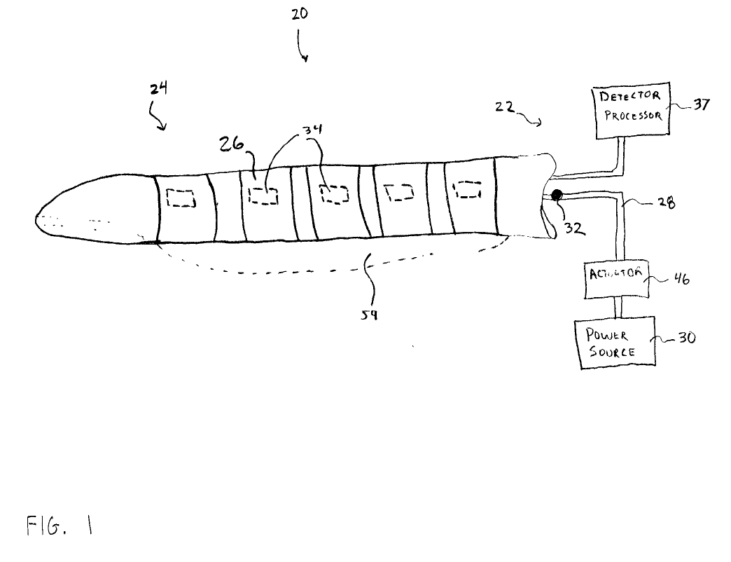

FIG. 1 illustrates a catheter incorporating the present invention. Catheter 20 includes a proximal portion 22 and a distal portion 24. An array of heat delivery electrodes 26 can be disposed on the distal portion 24 of the catheter. A movable conductor or energy conduit 28 is positioned within an inner lumen of the catheter 20 to move between the electrodes in the catheter. A distal tip of the conductor 28 can contact an inner surface of the electrodes such that an energy or current can be delivered from a power source 30 to the selected electrode. An external fiducial 32 may be positioned outside the body that is visible to the user to help the user accurately determine the position of the conductor 28 relative the electrodes 26.

The power source 30 can drive an energy through the electrodes 26 that is sufficient to deliver a therapeutic treatment (e.g. RF, AC, DC) to the vulnerable plaque or other target tissue. Depending on the type of tissue being treated, the resistivity of the electrodes and/or the amount of current can be selected to deliver a proper amount of heat to the material adjacent the electrode. The thermal conductivity of the electrode will distribute the heat generated by the current over the outer surface of the electrode and into the material adjacent the electrode. The resistivity of the electrode can be tailored for a desired ratio of heat directed into the tissue adjacent the electrode versus elsewhere in the catheter; such as ratio can be 2:1, 5:1, 10:1, or more.

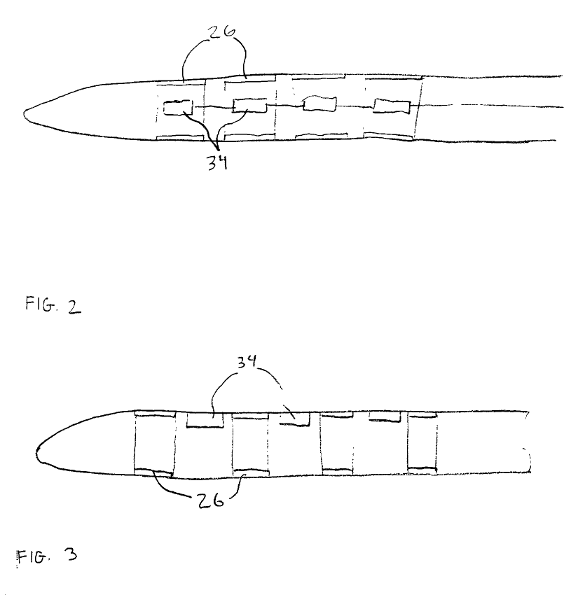

In exemplary embodiments, the catheter will include an array of position sensitive detectors 34 disposed on the distal portion of the catheter and coupled to a processor 37 for detecting marked lesions 36 in the body lumen 38. As shown in one embodiment in FIG. 2, the electrodes 26 are tubular and the detectors 34 are positioned inside the catheter body 22 and tubular electrodes 26. In another embodiment illustrated in, FIG. 3, the detectors 34 can be positioned interspersed between the individual electrodes. Because the detectors are position sensitive, the user will be able to determine the location of the lesion relative to the detectors and electrodes. Consequently, the user can activate the heating electrode(s) adjacent the lesion, while reducing heat delivery to the adjacent tissue. In some embodiments, the catheter 20 may include an inner sleeve (not shown) or outer sleeve (not shown) for housing the detectors 34 and electrodes 26.

The array of detectors 34 can be used to detect radiopharmaceuticals, optical fluorescence labeling, para- or ferromagnetic agents, and the like. If the detectors detect are configured to detect radiopharmaceuticals, the detectors can be positioned inside the heating electrodes. Alternatively, if the detectors are adapted to detect the other labels, the detectors can be interspersed amongst the heating electrodes and disposed along an external surface of the catheter. In an exemplary embodiment, radiopharmaceuticals will be delivered to the body lumen and bound to vulnerable plaque within the body lumen.

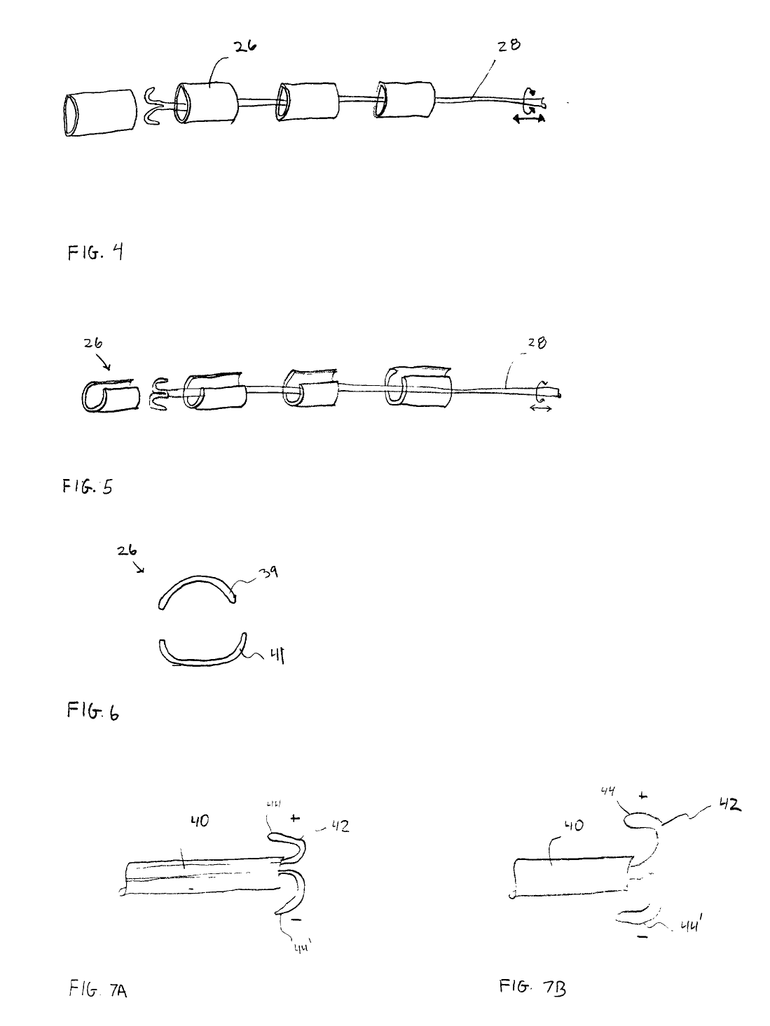

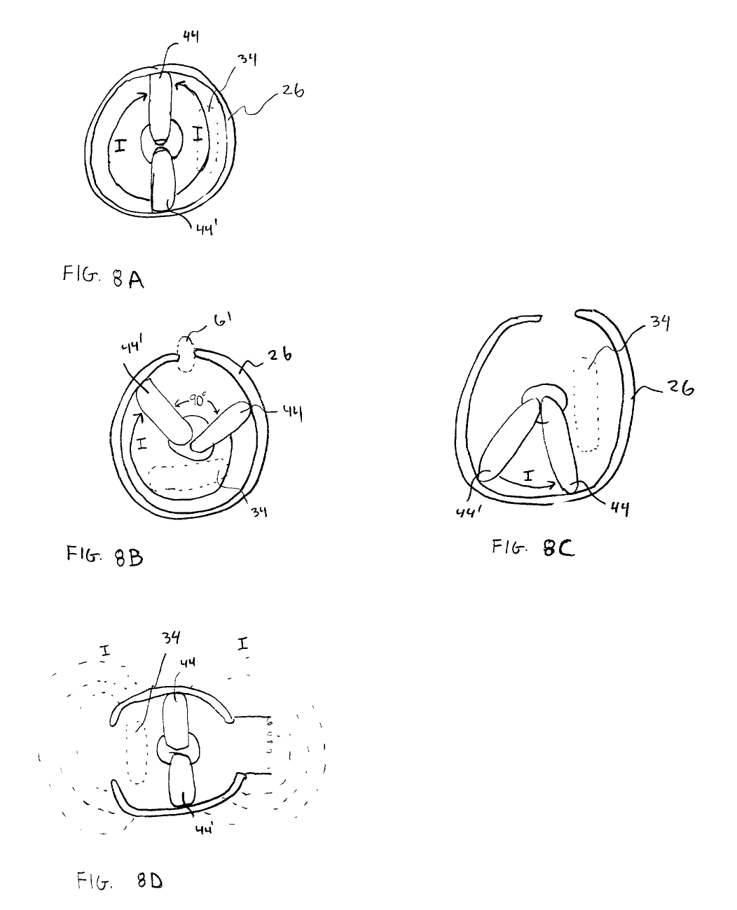

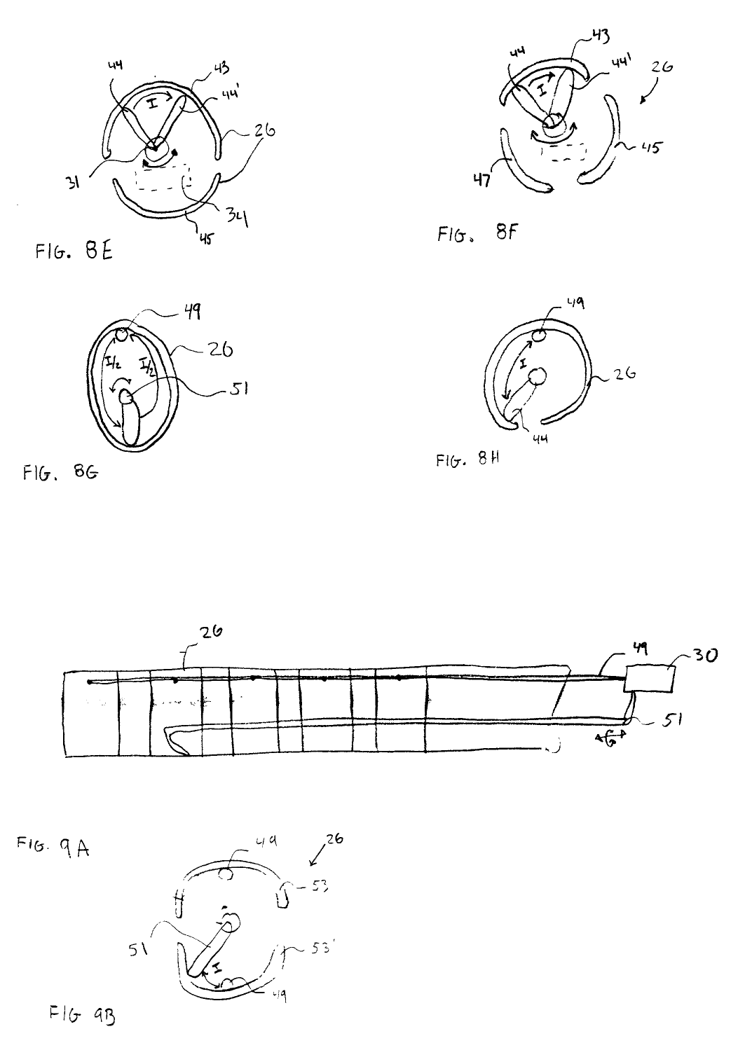

The electrodes 26 can be disposed along an inside or outside surface of the catheter 20 by coating or otherwise providing a conductive layer that is broken so as to form discrete sections along a portion of the catheter body. In the embodiments illustrated in FIGS. 4 and 5, the heat delivery electrodes 26 can be aligned along a longitudinal axis of the catheter 20. The electrodes 34 can be a tubular or cylindrical shape so as to be able to deliver heat around 360° of the catheter body. In a particular embodiment shown in FIG. 5, an insulating break can be formed or etched into the tubular electrode 34 to reduce the number of current paths through the electrode (See FIGS. 8A to 8H). It should be appreciated however, that the electrodes of the present invention can take a variety of forms. For example, the electrode can be elliptical, expandable, hinged, flattened, or the like.

Referring now to FIGS. 6 and 8D, the electrodes of the present invention can also be an RF antenna 26. The RF antenna 26 will typically include a positive portion 39 and negative portion 41 and be separated along two lengths. The current will be driven between the two separated RF antennas to heat the target tissue (e.g., vulnerable plaque) positioned adjacent the selected electrode 26.

The electrodes 26 can be coupled to the power source 30 through a movable conductor or energy conduit. The conductor can be moved axially to contact axially spaced electrodes and rotated about its longitudinal axis to contact azimuthally spaced electrodes (if any). The movable conductor 28 can reduce the number of transmission lines extending through the catheter body so as to allow the catheter to maintain a small diameter and flexibility. Consequently, the catheters of the present invention may be advanced through small and tortuous body lumens that conventional catheters cannot reach. In exemplary embodiments, the catheters of the present invention include a lumen (not shown) that is sized to receive a guidewire. The guidewire can be positioned adjacent the movable conductor, opposite the electrode array, positioned in an external lumen, in a rapid exchange configuration, or the like.

FIGS. 7A to 7B illustrate some exemplary movable conductors of the present invention. The conductors 28 are typically composed of a wire that has a body 40 and a distal tip 42. The distal tip can be bent outward to form sliding contacts 44, 44′. The sliding contacts 44, 44′ contact and bias against an inner surface of the electrodes to create a connection between the electrode and the power supply. The position of the sliding contacts 44, 44′ within the catheter can be accurately determined by viewing the position of suitable fiducials 32 (FIG. 1). A proximal end of the conductor 28 can be moved through an inner lumen of the catheter until its sliding contact 44, 44′ contacts the desired cylindrical electrode 26. The power source 30 can then drive a current through a path in the cylindrical electrode 26 to heat the vulnerable plaque or other target tissue adjacent the selected electrode. The sliding contacts can be driven manually or mechanically under a computer control.

As shown in FIG. 7B, the conductor 28 can comprise a double-strand wire that has a two sliding contacts 44, 44′. As shown in FIG. 7A, the conductor 26 can also comprise a coaxial wire having two sliding contacts 42. Some exemplary configurations of the sliding contacts 44 are illustrated in FIGS. 8A to 8G. As illustrated, the sliding contacts 44 can be disposed at different angles. A cylindrical electrode and a conductor 28 with a separation of approximately 180° splits the current in along two paths (FIG. 8A) and provides less efficient heating, but is easier to manufacture and is less prone to rotational loss of alignment. For embodiments using a split cylinder configuration, it is preferable to have a separation of less than 180° so as to reduce the chance of rotational loss of alignment. For example, the electrodes can be positioned 90° degrees apart or less from each other (FIG. 8B), 45° degrees apart or less from each other (FIG. 8C), 135 degrees apart or less, or the like.

FIGS. 8E to 9B illustrate yet other alternative electrode and conductor configurations. To better control the azimuthal delivery of heat, the electrode 26 can be broken along one or more longitudinal lengths. As shown in FIG. 8E the electrodes can be broken along two lengths so that the current will be delivered between the sliding contacts 44 only over electrode portion 43. Thus no heat will be delivered to separated electrode portion 45. To deliver heat to electrode 45, the conductor 26 can be rotated about longitudinal axis 31 until the sliding contacts 44, 44′ contact electrode 45. As shown in FIG. 8F, the electrode can be broken into three portions 43, 45, 47 such that the heating can be delivered to an even more localized portion of the body lumen. It should be appreciated that the electrodes of the present invention can be broken into more than three discrete portions, if desired.

Referring now to FIG. 9A, the electrodes 26 can be coupled to power source 30 through a fixed conductor 49 and a sliding conductor 51. Once the marked lesion is located in the body lumen, the sliding conductor 51 can be moved to the electrode 26 adjacent the lesion and the current can be delivered to the electrode through the sliding contact. As shown in FIGS. 8G and 8H, azimuthal control of the heat deliver can be controlled through the rotational alignment of the sliding contact 44 of conductor 51. For example, as shown in FIG. 8H, to heat a left portion of the body lumen, the sliding conductor 51 can be positioned so that the current will flow through substantially the left portion of the electrode 26. Alternatively, to heat a smaller portion of the body lumen, the sliding conductor can be rotated around longitudinal axis 31 closer to the fixed conductor 49 so that the current is delivered through a smaller portion of electrode 53. It should be appreciated that in some embodiments it may be desirable to rotate the entire catheter to control the azimuthal delivery of the heat treatment.

The catheters of the present invention may comprise two or more fixed conductors to better control the azimuthal delivery of heat. For example, as shown in FIG. 9B, each cylindrical electrode can be broken into more than two portions such that each electrode is contacted by a fixed conductor 49, 49′. Movement of the sliding contact 51 into contact with the desired electrode portion 53′ can deliver a current to the desired portion of the body lumen.

For solid cylindrical electrodes, the current follows two current paths and generally can deliver heat to the entire target portion of the body lumen. In contrast, for the broken cylindrical electrode there is just one current path and localized heating of the body lumen. The degree of localization of the heat will typically depend on the conductivity of the external medium and the thickness of the pad, which because of its thermal conductivity will tend to distribute heat over its surface.

For both the solid cylindrical electrode and the broken cylindrical electrode, the energy dissipated per unit length will depend on the resistance of the unit length. For a solid cylinder electrode, the current will split and flow through two resistors (i.e., two current paths) but at a lower current. The energy dissipated into heat will depend on RI². Thus, for the solid cylinder electrode (assuming a symmetric configuration), the heat dissipation will equal 2R(J/2)² or RI²/2, or about half the heat dissipation compared to the broken cylinder electrode made of the same material (i.e., RI²).

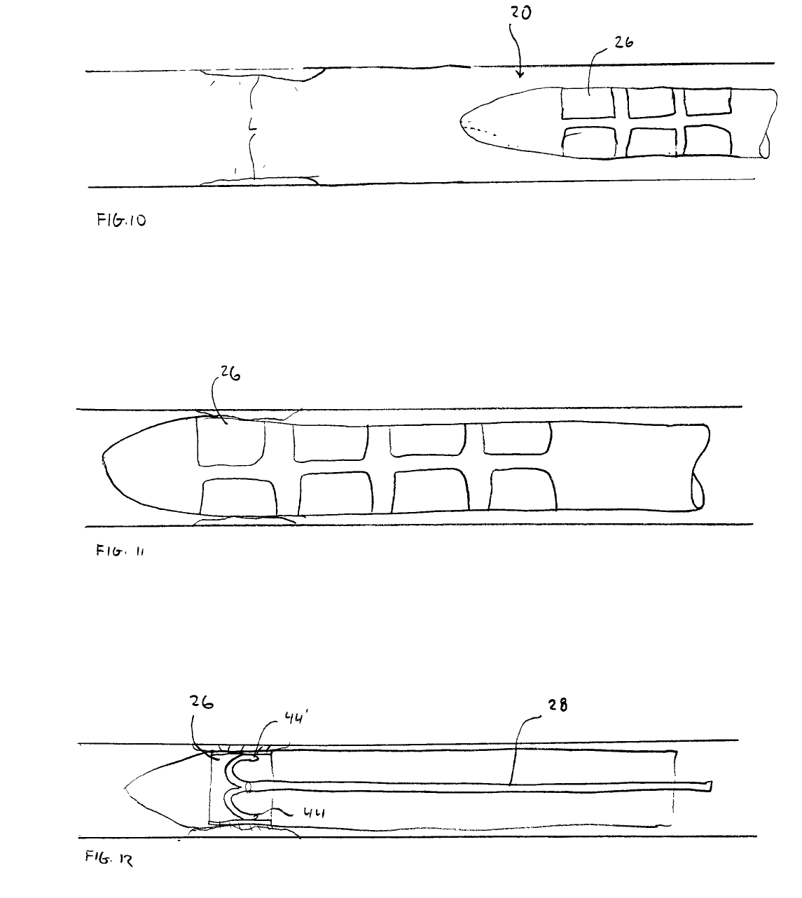

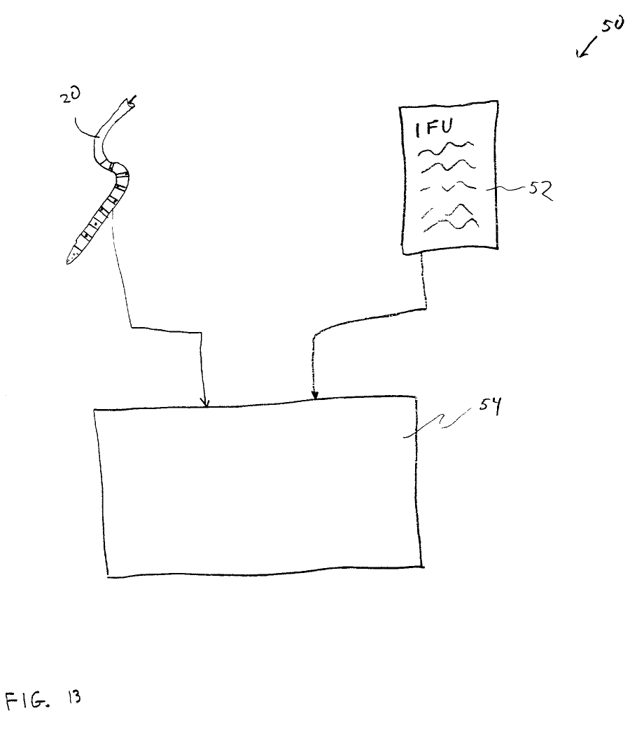

In use, the catheter is positioned at the target site within the body lumen (FIG. 10). The catheter will typically be advanced through the body lumen over a guidewire (not shown) to the target site. Once at the target site, the detectors are used to localize the lesion L (e.g., vulnerable plaque) (FIG. 11). Because the detectors are positioned adjacent the electrodes (e.g. interspersed, within, parallel, overlaid, or the like), the catheter does not have to be moved to deliver the therapeutic heat treatment. Thus, the catheter can be maintained in a substantially stationary position after the vulnerable plaque has been localized.

To activate the electrode adjacent the lesion (e.g. radiolabeled vulnerable plaque), the conductor is moved to the selected electrode in the array of electrodes (FIG. 12). A mechanical actuator and/or an electronic actuator 46 (FIG. 1) can be coupled to the conductor to move the sliding contacts 44 into engagement with the selected electrode. It should be appreciated, however, that the conductor can be manually moved to the electrode to deliver the heating current to the electrode. The position of the sliding contacts can be accurately determined by use of fiducials on the catheter body. The power source can then drive a current through the conductor to the selected electrode to heat the lesion.

Optionally, the catheters of the present invention can include means for pressing the electrodes against the body lumen wall. The pressing means can include a balloon or other expandable element disposed along an outside surface of the catheter body that can be inflated to move the distal portion of the catheter (and electrodes) against the body lumen (shown as 59 in phantom in FIG. 1). In other embodiments, a balloon, spring means, or other expandable device can be disposed within the electrodes 26 so as to expand the electrodes against the body lumen wall. In a specific embodiment, a balloon or spring 61 can be positioned with a longitudinal break in the electrode to press the electrodes outward against the body lumen (FIG. 8B).

During transit through the body lumen, the detectors can optionally be in a “gross count mode” to determine if marked lesions are in the body lumen surrounding the catheter. Once it is determined that a lesion is present, (e.g., a threshold gross count is reached), the detectors can be switched to an imaging mode to localize the lesion.

As illustrated in FIG. 13, the present invention further provides kits 50 including catheters 20, instructions for use 52 and a package 54. The catheters will generally be those as described above and the instruction for use (IFU) will set forth any of the methods described above. The package may be any conventional medical device packaging, including pouches, trays, boxes, tubes, or the like. The instructions for use will usually be printed on a separate piece of paper, but may also be printed in whole or in part on a portion of the packaging. Optionally, the kits can include a guidewire, radiopharmaceuticals for bonding to the unstable plaque, or the like.

As will be understood by those of skill in the art, the present invention may be embodied in other specific forms without departing from the essential characteristics thereof. For example, in some configurations the detector array can be mounted on the conductor and moved and rotated with the conductor. Once the detector array localizes a marked lesion, the a therapeutic current can be delivered to the electrode adjacent the localized lesion.

In another alternative embodiment, the electrode array of the present invention can deliver current to more than one electrode simultaneously. For example, the catheter may include a plurality of movable conductors so as to couple more than one electrode to a power supply.

In further alternative embodiments, the heat electrodes or applicator pads can be heated by a fiberoptic line that delivers visible or infrared light. Such an embodiment can be controlled longitudinally by position and azymuthally by rotation. The heating pads of this embodiment can be thermally conductive and optically absorbent. A similar method can be used to heat the pads through an ultrasonic energy-delivery system. In yet other embodiments, the applicator pads can be heated by a sliding internal resistor at the end of a coaxial or two-strand wire. The resistor can also slide on the outside of the catheter, with its own applicator pad, which can cover a fill or partial area in the azimuthal direction.

Accordingly, the foregoing description is intended to be illustrative, but not limiting, of the scope of the invention which is set forth in the following claims.

What is claimed is:

1. A heat delivery catheter comprising:
a catheter body comprising a proximal portion and a distal portion;
an electrode array disposed at the distal portion of the catheter body; and
an energy conduit positionable within the catheter body to energize individual electrodes of the electrode array so as to selectively deliver energy from a power supply to at least one selected electrode in communication with the energy conduit.

2. The catheter of claim 1 further comprising a detector array disposed at the distal portion of the catheter body, wherein the detector array can detect a marked lesion in the body lumen.

3. The catheter of claim 2 wherein the detector array comprises a plurality of detectors, wherein the detectors are interspersed between the array of electrodes.

4. The catheter of claim 2 wherein the electrodes of the electrode array are tubular and wherein the detector array comprises a plurality of detectors disposed within the tubular electrodes.

5. The catheter of claim 1 wherein the electrodes are tubular.

6. The catheter of claim 5 wherein the tubular electrodes comprise at least one longitudinal insulating break.

7. The catheter of claim 5 wherein the individual electrodes of the electrode array are axially spaced apart along an axis of the catheter body.

8. The catheter of claim 1 wherein the individual electrodes of the electrode array are azimuthally spaced apart along the catheter body.

9. The catheter of claim 1 wherein the individual electrodes of the electrode array are azimuthally and axially spaced apart along an axis of the catheter body.

10. The catheter of claim 1 wherein the energy conduit comprises a coaxial wire or a two strand wire.

11. The catheter of claim 10 wherein a distal tip of the energy conduit comprises at least one sliding contact.

12. The catheter of claim 11 wherein the sliding contact is configured to press against an interior surface of the selected electrode.

13. The catheter of claim 11 wherein the energy conduit comprises two sliding contacts that are azimuthally spaced 180° apart.

14. The catheter of claim 13 wherein the sliding contacts are positioned less than 180° apart.

15. The catheter of claim 13 wherein the sliding contacts are positioned approximately 135° apart or less.

16. The catheter of claim 13 wherein the sliding contacts are positioned approximately 90° apart or less.

17. The catheter of claim 13 wherein the sliding contacts are positioned approximately 45° apart or less.

18. The catheter of claim 1 further comprising a fixed conductor coupled to each of the electrodes in the electrode array.

19. A system comprising the catheter of claim 1 in combination with a power supply which delivers a DC current, AC current, or an RF current.

20. The catheter of claim 1 wherein the energy conduit comprises an optical fiber.

21. A system comprising the catheter of claim 20 in combination with a power supply which delivers optical or infrared light.

22. A system comprising the catheter of claim 1 in combination with an ultrasonic power supply.

23. The catheter of claim I wherein the electrode array is disposed within an inner lumen of the catheter body.

24. The catheter of claim 1 wherein the electrode array is disposed over an exterior surface of the catheter body, wherein the individual electrodes of the electrode array comprise inner contacts.

25. A method of delivering heat to a body lumen, the method comprising:
positioning a catheter comprising an electrode array in the body lumen; and
selectively delivering energy to fewer than all of the electrodes in the electrode array to heat a target region of the body lumen.

26. The method of claim 25 wherein selectively delivering energy comprises positioning a movable energy conduit within the catheter to selectively energize at least one selected electrode of the electrode array.

27. The method of claim 26 wherein positioning a movable energy conduit comprises contacting a single electrode.

28. The method of claim 26 wherein positioning a movable energy conduit comprises sliding the energy conduit within an inner lumen of the catheter body.

29. The method of claim 26 wherein positioning a movable energy conduit comprises contacting the energy conduit along an inner surface of the electrode.

30. The method of claim 26 wherein positioning comprises axially positioning the movable energy conduit adjacent the selected electrode(s) of the electrode array.

31. The method of claim 26 wherein positioning comprises azimuthally positioning the movable energy conduit adjacent the selected one(s) of the electrode array.

32. The method of claim 26 wherein positioning comprises azimuthally and axially positioning the movable energy conduit adjacent the selected electrode(s) of the electrode array.

33. The method of claim 26 wherein positioning further comprises contacting sliding contacts of the energy conduit against the electrode.

34. The method of claim 25 further comprising:
marking a lesion with a radiopharmaceutical; and
detecting the marked lesion in the body lumen using a detector on the catheter.

35. The method of claim 34 wherein the detector comprises a plurality of detectors, wherein detecting comprises positioning a plurality of detectors adjacent the electrode array.

36. The method of claim 34 wherein the detector comprises a plurality of detectors wherein detecting further comprises overlaying the plurality detectors over the electrode array.

37. The method of claim 25 further comprising maintaining the position of the catheter during detecting the lesion and delivering energy to the electrode(s).

38. The method of claim 25 wherein positioning comprises advancing the catheter through the body lumen.

39. The method of claim 25 wherein selectively delivering comprises creating at least one current path through the selected electrode.

40. The method of claim 25 wherein selectively delivering comprises driving an AC current, DC current, optical light, ultrasonic energy, or RF current to the selected electrode.

41. The method of claim 25 further comprising tracking the position of the energy conduit within the catheter.

42. The method of claim 25 further comprising generating a 10:1 ratio, a 5:1 ratio, or a 2:1 ratio of energy directed into the electrode and energy dissipated along the catheter.

43. The method of claim 25 wherein selectively delivering comprises heating only a selected azimuthal portion of the body lumen.

44. The method of claim 25 further comprising biasing at least a portion of the electrode array against the body lumen.

45. A catheter for heating vulnerable plaque in a body lumen, the system comprising:
a catheter body comprising a distal portion and a proximal portion;
a detector array disposed at the distal portion of the catheter body;
an electrode array disposed at the distal portion of the catheter body; and
a energy conduit positionable within the catheter body to selectively energize individual electrodes of the electrode array so as to selectively deliver energy from a power supply to at least one selected electrode in communication with the energy conduit.

46. The catheter of claim 45 wherein the vulnerable plaque is labeled with a radiopharmaceutical, wherein the detector array comprises a plurality of position sensitive radiation detectors.

47. The catheter of claim 45 wherein the movable energy conduit comprises a distal tip having two sliding contacts.

48. The catheter of claim 47 wherein the two sliding contacts are disposed at an angle of approximately 180° or less, 90° or less, or 45° or less apart from each other.

49. A system comprising the catheter of claim 45 in combination with a power supply which delivers a optical energy, ultrasonic energy, DC current, AC current, or an RF energy.

50. The system of claim 45 wherein the energy conduit comprises a two strand conductor or a coaxial conductor.

51. The system of claim 45 wherein the energy conduit comprises an optical fiber.

52. The catheter of claim 45 wherein the catheter body comprises an outer sleeve and an inner sleeve, wherein the detector array is disposed on one of the inner sleeve and outer sleeve and the electrode array is disposed on the other of the inner and outer sleeve.

53. The catheter of claim 45 wherein the electrode array comprises a plurality of semiconductor radiation detectors.

54. The catheter of claim 45 wherein the electrode comprises at least one scintillator and a plurality of optical detectors.

55. The catheter of claim 45 wherein the electrode array comprises a delay line.

56. The catheter of claim 45 further comprising a fixed conductor coupled to the individual electrodes in the electrode array, wherein the energy flows between the positionable energy conduit and the fixed conductor.

57. The catheter of claim 45 further comprising means for biasing the electrode against the body lumen.

58. A method of treating vulnerable plaque in a body lumen, the method comprising:
marking the vulnerable plaque with a radiopharmaceutical;
imaging the marked vulnerable plaque in the body lumen; and
heating the marked vulnerable plaque in the body lumen.

59. The method of claim 58 wherein imaging comprises positioning a catheter having at least one detector and a plurality of electrodes into the body lumen.

60. The method of claim 58 wherein heating comprises selectively delivering energy to fewer than all of the electrodes to heat a target region of the body lumen.

61. The method of claim 60 wherein selectively delivering energy comprises positioning a movable energy conduit within the catheter to selectively contact selected electrode(s).

62. A kit comprising:
a catheter comprising a catheter body having an electrode array and a energy conduit positionable within the catheter body to selectively contact individual electrodes of the array so as to selectively deliver a current from a power supply to at least one selected electrode in communication with the energy conduit;
instructions for use comprising positioning a catheter in the body lumen, moving the energy conduit to the selected electrode and delivering a current through the energy conduit to the selected electrode; and
a package for holding the catheter and the instructions for use.

63. The kit of claim 57 further comprising a guidewire.

64. The kit of claim 57 wherein the catheter further comprises an array of detectors.

65. The kit of claim 57 wherein the energy conduit comprises a sliding contact.

66. A method of treating vulnerable plaque in a body lumen, the method comprising:
positioning an electrode array adjacent the vulnerable plaque in the body lumen;
pressing at least a portion of the electrode array against the vulnerable plaque; and
delivering a therapeutic treatment to fewer than all of the electrode(s) in the electrode array to heat the vulnerable plaque in the body lumen.

67. The method of claim 66 wherein delivering comprises moving a movable energy conduit into contact with an electrode adjacent the vulnerable plaque.

68. The method of claim 66 wherein delivering comprises transmitting the current to a selected azimuthal portion of the electrode.

69. A catheter comprising:
a catheter body comprising a proximal portion and a distal portion;
an electrode array disposed at the distal portion of the catheter body; and
movable delivery means for selectively delivering energy from a power supply to at least one selected electrode in communication with the delivery means.

70. The catheter of claim 11 wherein the sliding contact comprises a resistor connected to a coaxial or two-strand wire.

71. The catheter of claim 11 wherein the sliding contact comprises a resistor connected to a single wire, a sliding contact from the resistor to a heating pad, and a common return path through the pad.
